SEQUENCE LISTING (9 sequences)

SEQ ID NO: 1 - DNA, 27 bp, Artificial Sequence
  CSN-N1 PRIMER
aaaactgcagcattttatgtagtaagc

SEQ ID NO: 2 - DNA, 23 bp, Artificial Sequence
  CSN-C1 PRIMER
ccggaattcgtatgctaattccc

SEQ ID NO: 3 - DNA, 33 bp, Artificial Sequence
  BAM-FP PRIMER
ggatcctatgtatccgctcatgagacaataacc

SEQ ID NO: 4 - DNA, 38 bp, Artificial Sequence
  DEL-RP PRIMER
ggcattctgcactcttcacctagatcctttttgatcag

SEQ ID NO: 5 - DNA, 43 bp, Artificial Sequence
  BSG-SF PRIMER
cgggatccttctgcactatgtatccgctcatgagacaataacc

SEQ ID NO: 6 - DNA, 49 bp, Artificial Sequence
  BSG-SR PRIMER
nnnacgtcaattacggatcctgcactcttcacctagatcctttttgatc

SEQ ID NO: 7 - DNA, 46 bp, Artificial Sequence
  BSG-IF PRIMER
cgggatccttgcactgcactatgtatccgctcatgagacaataacc

SEQ ID NO: 8 - DNA, 41 bp, Artificial Sequence
  BSG-DF PRIMER
gctacgcactgcactatgtatccgctcatgagacaataacc

SEQ ID NO: 9 - DNA, 38 bp, Artificial Sequence; identical to SEQ ID NO: 4
  BSG-DR PRIMER

(19) United States
(12) Patent Application Publication
(10) Pub. No.: US 2005/0074892 A1
(43) Pub. Date: Apr. 7, 2005

(54) Transposon-mediated random codon-based mutagenesis

(76) Inventors: Si-Hyoung Lee, Jinju-si (KR); Eun-Sun Wang, Hadong-gun (KR); Eun-Jung Kim, Sacheon-si (KR); John YJ. Jeon, Seoul (KR); Yong-Chul Shin, Jinju-si (KR)

Correspondence Address: ANDERSON, KILL & OLICK, P.C., 1251 AVENUE OF THE AMERICAS, NEW YORK,, NY, 10020-1182, US

(21) Appl. No.: 10/613,855

(22) Filed: Jul. 3, 2003

(30) Foreign Application Priority Data
Jul. 4, 2002 (KR) 2002-0038563

(51) Int. Cl. C12N 15/74

(52) U.S. Cl. 435/473

(57) ABSTRACT

A method for evolving a polypeptide or a polynucleotide coding therefor, which comprises preparing a library of mutant polynucleotides through transposon-mediated random substitution, insertion or deletion of a multiple of three nucleotides on a polynucleotide coding for a target protein, expressing the mutant polynucleotides in a host cell and screening for a polypeptide having a desired property.

### FIELD OF THE INVENTION

The present invention relates to a transposon-mediated random codon-based mutagenesis for the directed evolution of a protein. More specifically, it pertains to a method for evolving a polypeptide or a polynucleotide encoding same, which comprises preparing a library of mutant polynucleotides through transposon-mediated random substitution, insertion or deletion of a multiple of three nucleotides on a polynucleotide coding for a target protein, expressing the mutant polynucleotides in a host cell and screening for a polypeptide having a desired property.

### BACKGROUND OF THE INVENTION

Genetic information is eventually decoded into proteins which perform most of the vital functions in living organisms. As one of important biological macromolecules, a protein not only serves as a component of cells but also participates in a specific biochemical reaction with a high specificity.

The function of a protein is determined by the structure which is divided into four levels; primary, secondary, tertiary and quaternary structures. Since the primary structure of a protein, i.e., the sequence of amino acids, contains the information regarding the shape and the function thereof, the whole structure or function of the protein can be changed by a mutation of even one amino acid residue (Shao, Z. and Arnold F. H., Curr. Opin. Struct. Biol. 6:513-518, 1996).

Owing to the rapid development of genetic technologies, it has become possible to clone any gene coding for a protein and produce the protein on a large scale by employing the cloned gene. Further, there have been designed various methods to introduce a high frequency of mutations in a gene by in vitro mutagenesis, thereby obtaining a protein evolved for the desired purpose.

Recently, a directed evolution technique is widely used as a potent tool for obtaining a useful mutant protein having a desired property, which comprises preparing a library of mutated genes from a target gene coding for the target protein and screening the proteins encoded thereby to obtain the mutated proteins. This technique mimics the natural evolution including mutation and selection processes occurring over a long period of time in nature, but, the desired mutant protein can be obtained thereby in a short time (Kuchner, O. and Arnold F. H., Trends Biotechnol. 15:523-530, 1997; Sutherland, J. D., Curr. Opin. Chem. Biol. 4:263-269, 2000; Bornscheuer, U. T. and Pohl, M., Curr. Opin. Chem. Biol. 5:137-143, 2001).

Exemplary techniques in widespread use for preparing mutant polynucleotides for the directed evolution include site-directed mutagenesis (Sambrook, J. and Russell, D. W., Molecular Cloning: A Laboratory Manual, 3rd ed., Cold Spring Harbor Laboratory Press, N. Y., 2001), cassette mutagenesis (Arkin, A. and Youvan, D. C., Proc. Natl. Acad. Sci. USA 89:7811-7815, 1992; Delagrave et al., Protein Engineering 6:327-331, 1993; Goldman, E. R. and Youvan, D. C., Bio/Technology 10:1557-1561, 1992), saturation mutagenesis (U.S. Pat. No. 6,171,820 and U.S. Pat. No. 6,238,884), error-prone PCR method (Leung, D. W. et al., Technique 1:11-15, 1989; Caldwell, R. C. and Joyce, G. F., PCR Methods and Applications 2:28-33, 1992; Gramm, H. et al., Proc. Natl. Acad. Sci. USA 89:3576-3580, 1992), and chemical mutagenesis (Myers, R. M. et al., Science 229:242-247, 1985; Walton, C. et al., Directed Mutagenesis: A practical approach (ed. M. J. McPherson), 135-162, IRL Press, Oxford, United Kingdom, 1991).

Among the above mutagenesis techniques, the site-directed mutagenesis, saturation mutagenesis and cassette mutagenesis are used for mutation at a specific site of a protein, and error-prone PCR method and chemical mutagenesis are used for mutation at a random site.

Site-directed mutagenesis replaces nucleotides of a desired site with a synthetically mutated oligonucleotide. However, there are limitations of the method in that it requires prior knowledge of the amino acid sequence of a target protein and the function of the site to be mutated. Further, this method is not appropriate for the systematic mutation of each and every amino acid of a polypeptide, since this requires separate synthesis of individual oligonucleotide necessary for each mutation.

The saturation mutagenesis employing random oligonucleotides is used for substituting an amino acid with any possible amino acid. Cassette mutagenesis is used for substituting a specific site of a desired DNA with a synthetic oligonucleotide cassette containing altered nucleotides. However, these methods are also inappropriate for the systematic mutation of each and every amino acid of a polypeptide, since they require a burden of high cost, long time and excessive work.

Currently, error-prone PCR method is widely used for constructing a mutant DNA library of a gene. In this method, a mutant library is prepared by controlling the polymerization conditions to change the error rate of a polymerase. However, the error-prone PCR method has a problem in that it is difficult to control the error rate appropriately to obtain a desired frequency of mutation. Moreover, the frequency of co-occurrence of more than one base substitution within a codon is too low, so that the number of mutant amino acids for a given amino acid residue is limited.

Chemical mutagenesis causes random mutation on a target DNA by treating the DNA with a compound such as NTG (N-methyl-N′-nitro-N-nitrosoguanidine) and hydroxylamine. However, this method also requires careful control of the error rate of DNA polymerase and the number of substituting amino acids for one amino acid residue is limited.

Meanwhile, scanning mutagenesis employing a transposon (Hallet, B. et al., Nucleic Acids Res. 25:1866-1867, 1997; Cao, Y. et al., J. Mol. Biol. 274:39-53, 1997; Hayes, F. et al., J. Biol. Chem. 272:28833-28836, 1997; Hayes, F. et al., Cancer Res. 60:2411-2418, 2000) has been used for the mutagenesis of a protein. Since this method comprises mere insertion of transposon to a target DNA and deletion thereof from the resulting DNA, most of the mutants produced thereby have an insertion of five amino acids encoded by ten nucleotides originating from the transposon and five nucleotides duplicated during the insertion of the transposon to the target DNA. Accordingly, this method is quite limitative as a tool for improving the properties of a protein.

A method for constructing a mutant DNA library without such drawbacks of the conventional methods would provides a powerful tool for the production of mutant proteins having improved properties.

The present inventors have endeavored to develop a simple and economical method for preparing a mutant protein library useful for the directed evolution of a protein, and have achieved a transposon-mediated random codon-based mutagenesis method which comprises preparing a library of mutant polynucleotides through transposon-mediated random substitution, insertion or deletion of nucleotides on a polynucleotide coding for a target protein, expressing the mutant polynucleotides in a host cell and screening a polypeptide having a desired property.

### SUMMARY OF THE INVENTION

Accordingly, it is an object of the present invention to provide a method for preparing a useful mutant protein having desired properties and a polynucleotide encoding same, which comprises preparing a library of mutant polynucleotides by random substitution, insertion or deletion of nucleotides in a polynucleotide encoding a target protein, and screening the mutant proteins expressed therefrom to obtain a mutant protein having a desired property.

Another object of the present invention is to provide a method for preparing a useful mutant protein having desired properties and a polynucleotide encoding same, which comprises preparing a polynucleotide having a plurality of mutations by introducing two or more mutated sequences identified in two or more mutant polynucleotides into one target polynucleotide.

A further object of the present invention is to provide a method for evolving a polypeptide and a polynucleotide encoding same, which comprises repeating the above mutagenesis methods with a polynucleotide having a plurality of mutations as a target polynucleotide.

In accordance with one aspect of the present invention, there is provided a random codon-based mutagenesis wherein a library of mutant proteins is prepared efficiently by random substitution, insertion or deletion of amino acid residues in a target protein.

### BRIEF DESCRIPTION OF THE DRAWINGS

The above and other objects and features of the present invention will become apparent from the following description of the invention, when taken in conjunction with the accompanying drawings, which respectively show:

FIG. 1: a schematic diagram illustrating an example of the construction of a substitution mutant library employing the inventive random mutagenesis method, wherein consecutive three nucleotides are substituted with ATG or CAT at a random site of the target DNA. Tn7 is employed as a transposon, and the first and the second cassettes have a cleavage site of BsgI, a class IIS restriction enzyme. Nucleotides of the cassette DNA are represented in italics. The underlined nucleotides in Step 1 are those of target DNA duplicated during the insertion of transposon into the target DNA;
↑ ↓: a cleavage site of the restriction enzyme
Kan^{r}: kanamycin resistance gene

FIG. 2: a schematic diagram illustrating an example of the construction of an insertion mutant library employing the inventive random mutagenesis method, wherein random consecutive three nucleotides are inserted into a random site of the target DNA. Tn7 is employed as a transposon, and the first and the second cassettes have a cleavage site of BsgI, a class IIS restriction enzyme. Nucleotides of the cassette DNA are represented in italics. The underlined nucleotides in Step 1 are those of target DNA duplicated during the insertion of transposon into the target DNA;
N: a mixture of A, G, T and C
↑ ↓: a cleavage site of the restriction enzyme
Kan^{r}: kanamycin resistance gene

FIG. 3: a schematic diagram illustrating an example of the construction of a deletion mutant library employing the inventive random mutagenesis method, wherein consecutive three nucleotides are deleted at a random site of the target DNA. Tn7 is employed as a transposon, and the cassette have a cleavage site of BsgI, a class IIS restriction enzyme. Nucleotides of the cassette DNA are represented in italics. The underlined nucleotides in Step 1 are those of target DNA duplicated during the insertion of transposon into the target DNA; and
↑ ↓: a cleavage site of the restriction enzyme
Kan^{r}: kanamycin resistance gene

FIG. 4: nucleotide sequences of the recognition sites of class IIS restriction enzymes and cleavage sites thereof.

### DETAILED DESCRIPTION OF THE INVENTION

The present invention provides a method for evolving a polypeptide and a polynucleotide encoding same by random substitution of nucleotides, comprising the steps of:
1) inserting a transposon having restriction enzyme sites on both ends thereof into a random position of a double-stranded target DNA, introducing the resulting DNA into a circular DNA construct and cutting the transposon at the restriction enzyme sites to remove the transposon and obtain a linearized DNA construct containing two cut termini of the target DNA cut in one position;
2) deleting the nucleotides originating from the transposon and the nucleotides of the target DNA duplicated during the insertion of the transposon, at one cut terminus of the target DNA;
3) inserting a multiple of three substitutive nucleotides into one cut terminus of the target DNA subjected to deletion in Step 2, and deleting the nucleotides originating from the transposon and a multiple of three consecutive nucleotides of the target DNA at the other cut terminus of the target DNA obtained in Step 1;
4) subjecting both cut termini of the target DNA obtained in Step 3 to self-ligation to obtain a library of mutant DNA having substitutive nucleotides at a random position; and
5) expressing the resulting library in an appropriate host cell and selecting or screening the expressed polypeptides to obtain a mutant polypeptide having a desired property and a polynucleotide encoding same.

This method will be more easily understood in conjunction with an example illustrated in FIG. 1.

In this method, Step 2 may comprise the steps of: introducing a first cassette DNA into the cut position of the target DNA, digesting the cassette DNA with a restriction enzyme, and converting the cut terminus having nucleotides duplicated during the insertion of the transposon to a blunt end, thereby resulting in the deletion of the nucleotides originating from the transposon and the nucleotides of the target DNA duplicated during the insertion of the transposon.

Further, Step 3 may comprise the steps of: introducing a second cassette DNA containing a multiple of three consecutive substitutive nucleotides into the cut position of the DNA obtained in Step 2, digesting the second cassette DNA with a restriction enzyme, and converting both cut termini of the resulting DNA fragment to blunt ends, thereby resulting in the addition of the substitutive nucleotides into one cut terminus of the target DNA subjected to deletion in Step 2 and the deletion of the nucleotides originating from the transposon and a multiple of three consecutive nucleotides of the target DNA at the other cut terminus of the target DNA obtained in Step 1.

The present invention also provides a method for evolving a polypeptide and a polynucleotide encoding same by random insertion of nucleotides, comprising the steps of:
1) inserting a transposon having restriction enzyme sites on both ends thereof into a random position of a double-stranded target DNA, introducing the resulting DNA into a circular DNA construct and cutting the transposon at the restriction enzyme sites to remove the transposon and obtain a linearized DNA construct containing two cut termini of the target DNA cut in one position;
2) deleting the nucleotides originating from the transposon and the nucleotides of the target DNA duplicated during the insertion of the transposon, at one cut terminus of the target DNA;
3) inserting a multiple of three additional nucleotides into one cut terminus of the target DNA subjected to deletion in Step 2, and deleting the nucleotides originating from the transposon at the other cut terminus of the target DNA obtained in Step 1;
4) subjecting both cut termini of the target DNA obtained in Step 3 to self-ligation to obtain a library of mutant DNA having additional nucleotides at a random position; and
5) expressing the resulting library in an appropriate host cell and selecting or screening the expressed polypeptides to obtain a mutant polypeptide having a desired property and a polynucleotide encoding same.

This method will be more easily understood in conjunction with an example illustrated in FIG. 2.

In this method, Step 2 may comprise the steps of: introducing a first cassette DNA into the cut position of the target DNA, digesting the cassette DNA with a restriction enzyme, and converting the cut terminus having nucleotides duplicated during the insertion of the transposon to a blunt end, thereby resulting in the deletion of the nucleotides originating from the transposon and the nucleotides of the target DNA duplicated during the insertion of the transposon.

Further, Step 3 may comprise the steps of: introducing a second cassette DNA containing a multiple of three consecutive additional nucleotides into the cut position of the DNA obtained in Step 2, digesting the second cassette DNA with a restriction enzyme, and converting both cut termini of the resulting DNA fragment to blunt ends, thereby resulting in the insertion of the additional nucleotides into one cut terminus of the target DNA subjected to deletion in Step 2 and the deletion of the nucleotides originating from the transposon at the other cut terminus of the target DNA obtained in Step 1.

The present invention also provides a method for evolving a polypeptide and a polynucleotide encoding same by random deletion of nucleotides, comprising the steps of:
1) inserting a transposon having restriction enzyme sites on both ends thereof into a random position of a double-stranded target DNA, introducing the resulting DNA into a circular DNA construct and cutting the transposon at the restriction enzyme sites to remove the transposon and obtain a linearized DNA construct containing two cut termini of the target DNA cut in one position;
2) deleting the nucleotides originating from the transposon and the nucleotides of the target DNA duplicated during the insertion of the transposon, at one cut terminus of the target DNA, and the nucleotides originating from the transposon and a multiple of three consecutive nucleotides of the target DNA at the other cut terminus of the target DNA obtained in Step 1;
3) subjecting both cut termini of the target DNA obtained in Step 2 to self-ligation to obtain a library of mutant DNA having a deletion of nucleotides at a random position; and
4) expressing the resulting library in an appropriate host cell and selecting or screening the expressed polypeptides to obtain a mutant polypeptide having a desired property and a polynucleotide encoding same.

This method will be more easily understood in conjunction with an example illustrated in FIG. 3.

In this method, Step 2 may comprise the steps of: introducing a cassette DNA into the cut position of the target DNA, digesting the cassette DNA with a restriction enzyme, and converting both cut termini of the resulting DNA fragment to blunt ends, thereby resulting in the deletion of the nucleotides originating from the transposon and the nucleotides of the target DNA duplicated during the insertion of the transposon, at one cut terminus of the target DNA, and the deletion of the nucleotides originating from the transposon and a multiple of three consecutive nucleotides of the target DNA at the other cut terminus of the target DNA obtained in Step 1.

Further, the present invention provides a method for evolving a polypeptide and a polynucleotide encoding same, comprising the steps of:
1) preparing a library of mutant polynucleotides having a plurality of mutations by introducing two or more mutated sequences identified in two or more mutant polynucleotides, which are selected by the above random mutagenesis methods, into a target polynucleotide; and
2) expressing the library obtained in Step 1 in an appropriate host cell and selecting or screening the expressed polypeptides to obtain a mutant polypeptide having a desired property and a polynucleotide encoding same.

Moreover, the present invention provides a method for evolving a polypeptide and a polynucleotide encoding same, comprising repeating the inventive transposon-mediated random codon-based mutagenesis methods with the mutant polynucleotide having a plurality of mutations as a target polynucleotide.

As used herein, the term “codon-based mutagenesis” refers to the introduction of a mutation in a target polynucleotide by a multiple of three nucleotides, in order to induce a substitution, insertion or deletion of amino acids in a target protein encoded by the target polynucleotide, without causing a frame shift mutation. The number of consecutive nucleotides to be substituted, inserted or deleted may range from 3 to 30, preferably, 3 to 15.

Each step of the inventive methods for evolving a target polypeptide and polynucleotide by the transposon-mediated random codon-based mutagenesis is described in detail as follows.

In Step 1 of each of the inventive methods, a transposon having recognition sites for a restriction enzyme at both ends thereof is preferably used. Exemplary transposons for use in the present invention include Tn4430 having KpnI recognition sites(Hallet, B. et al., Nucleic Acids Res. 25:1866-1867, 1997), Tn7 having PmeI recognition sites(Biery, M. C. et al., Nucleic Acids Res. 28:1067-1077, 2000) and Mini-Mu having NotI recognition sites(Taira, S. et al., Mol. Microbiol. 34:736-744, 1999). These transposons may be introduced in the target DNA by conventional in vivo or in vitro transposition methods(Hallet, B. et al., supra; Biery, M. C. et al., supra; and Taira, S. et al., supra).

As can be seen from FIGS. 1 to 3, when the transposon is inserted in the target DNA, some nucleotides of the target DNA beside the insertion site are duplicated. For instance, in case of transposons Tn4430, Tn7 and Mini-Mu, 5 bp nucleotides of the target DNA are duplicated.

The target DNA may be in the form of a plasmid containing same or a DNA fragment. In case when the target DNA is a DNA fragment, the transposon may be inserted by employing the in vitro transposition method(Biery, M. C. et al., supra).

When the target DNA is included in a plasmid, the transposon may be inserted not only in the target DNA but also in the plasmid region other than the target DNA. In such case, a library of DNA constructs wherein a transposon is randomly inserted in the target DNA may be obtained by digesting the plasmid with appropriate restriction enzymes and collecting DNA fragments having a size corresponding to the target DNA plus transposon DNA. The DNA library obtained as above may be amplified by the polymerase chain reaction(PCR) employing appropriate primers for the target DNA. The resulting target DNA may be introduced in an appropriate plasmid to obtain a circular DNA construct.

Then, the DNA construct may be digested with a restriction enzyme of which cleavage sites are present at both ends of the transposon to remove the transposon therefrom. Consequently, as can be seen from FIGS. 1 to 3, the target DNA includes some nucleotides originating from the restriction enzyme cleavage site of the transposon, at its one cut terminus, and the target DNA, some nucleotides originating from the restriction enzyme cleavage site of the transposon and the nucleotides of the target DNA duplicated during the insertion of the transposon, at it's the other cut terminus.

In Step 2, some nucleotides at either or both cut termini of the resulting target DNA are removed depending on the type of mutagenesis to be desired. For this purpose, a first cassette DNA designed to have a recognition site for a restriction enzyme, preferably, a class IIS restriction enzyme, is inserted between both cut termini of the target DNA, and the resulting DNA construct is digested with the restriction enzyme to remove some nucleotides at either or both cut termini of the target DNA.

As used herein, the term “cassette DNA” refers to a DNA fragment used to be inserted between both cut termini of a target DNA for deleting or adding some nucleotides at the cut terminus of the target DNA.

The cassette DNA is a double-stranded DNA fragment consisting of a spacer and a restriction site for a restriction enzyme, preferably, a class IIS restriction enzyme, at either or both termini thereof. The spacer may consist of several to several thousands of base pairs, preferably, 20 to 3,000 bp. For the easy selection of the target DNA including the cassette DNA inserted therein, a marker gene such as an antibiotic resistance gene can be arranged in the spacer. The marker gene may be expressed by employing its own promoter or the promoter of the target DNA.

When the double-stranded cassette DNA is relatively short, it can be prepared by synthesizing several oligonucleotides constituting each strand of the double-stranded DNA and annealing the oligonucleotides at an appropriate condition. When the cassette DNA is very long due to the nucleotide sequences as long as several hundreds of base pairs, e.g., an antibiotic resistance gene, arranged in the spacer region, the cassette DNA may be prepared by amplifying the DNA to be arranged in the spacer region by the PCR method.

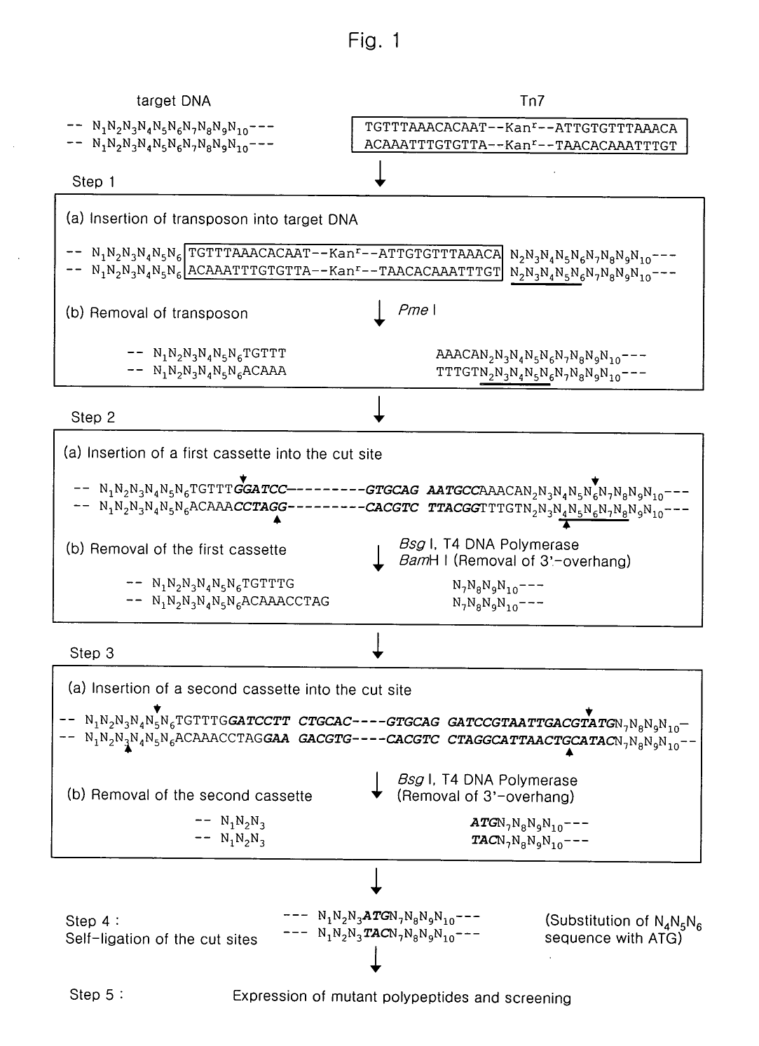

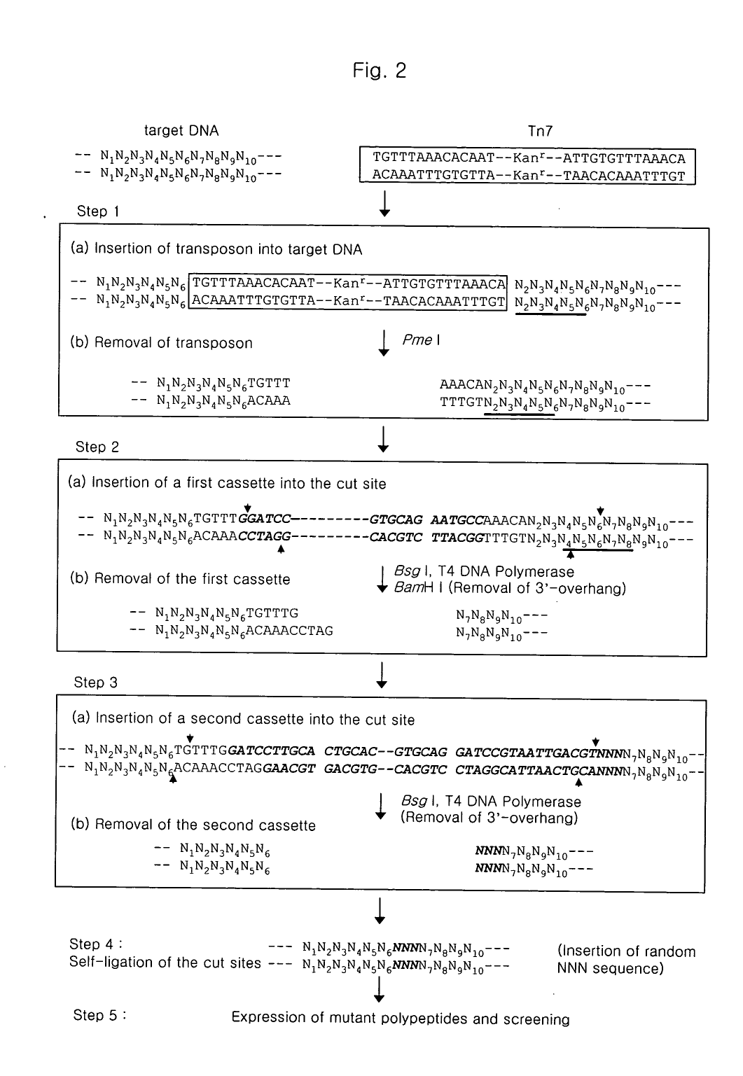

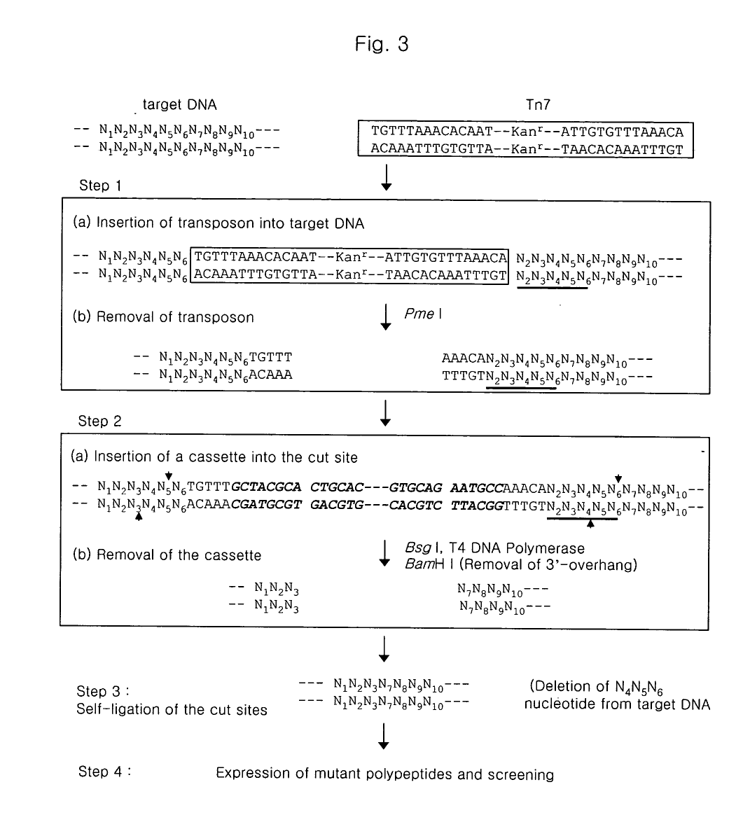

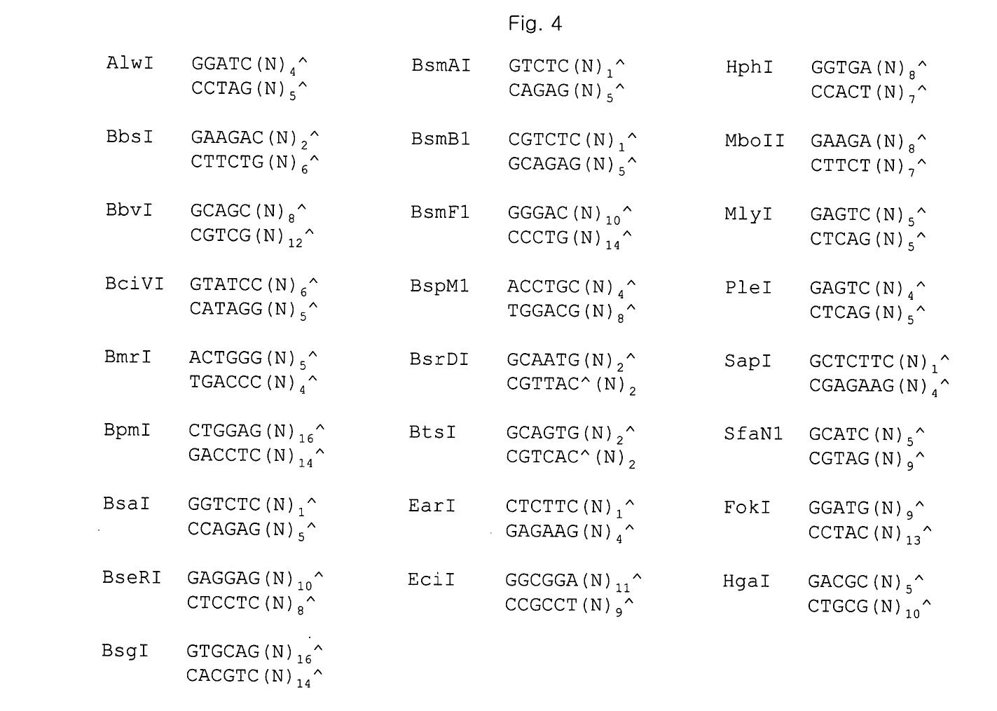

Meanwhile, class IIS restriction enzyme is a kind of DNA endonuclease, which recognizes a specific recognition site(See FIG. 4) and cleaves non-specific nucleotide sequences present at a constant distance downstream its recognition site. The cleavage site is away from the recognition site, e.g., by several to dozens of base pairs. Class IIS restriction enzyme generally produces a DNA fragment having a 5′-overhang or 3′-overhang.

The specific recognition site for the class IIS restriction enzyme should not be present in the target DNA and, if there is, the site should be mutated by site-specific mutagenesis or other appropriate class IIS restriction enzyme should be employed.

The resulting terminus made by digestion with the restriction enzyme, which has a 5′-overhang or 3′-overhang, may be converted to a blunt end, if necessary. In order to convert the cut region having 5′-overhang to a blunt end, an enzyme such as Klenow DNA polymerase, Mung Bean nuclease and SI nuclease may be used, wherein gap-filling reaction by Klenow DNA polymerase is preferred. Further, in order to convert the cut region having 3′-overhang to a blunt end, an enzyme such as T4 DNA polymerase, Klenow DNA polymerase, Mung Bean nuclease and S1 nuclease may be used, wherein removal of 3′-overhang by 3′→5′ exonuclease activity of T4 DNA polymerase is preferred.

In Step 3 of the codon-based substitution or insertion mutagenesis of a target DNA, a second cassette DNA is introduced in the cut region of the target DNA. The cassette DNA is defined as in Step 2.

Specifically, in case of the codon-based substitution mutagenesis of a target DNA, the cassette DNA may be designed to have a multiple of three substitutive nucleotides to be inserted in the target DNA and a cleavage site for an enzyme for the insertion of the substitutive nucleotides in the target DNA, at one cut terminus thereof; and a cleavage site for an enzyme for the deletion of nucleotides originating from the transposon and three consecutive nucleotides of the target DNA, at the other cut terminus thereof(See FIG. 1).

Further, in case of the codon-based insertion mutagenesis of a target DNA, the cassette DNA may be designed to have a multiple of three nucleotides to be inserted in the target DNA and a cleavage site of an enzyme for the insertion of the nucleotides in the target DNA, at one cut terminus thereof; and a cleavage site of an enzyme for the deletion of nucleotides originating from the transposon, at the other cut terminus thereof(See FIG. 2).

The cleavage site of an enzyme to be arranged in the second cassette DNA is preferably for a class IIS restriction enzyme.

The nucleotides to be arranged in the second cassette DNA for substitution or insertion may have a specific nucleotide sequence as illustrated in FIG. 1 or a random nucleotide sequence as in FIG. 2.

The second cassette DNA introduced in the target DNA may be removed from the target DNA by digesting it with an enzyme recognizing the restriction site thereof in the cassette DNA, while leaving some nucleotides to be inserted in the target DNA. In such case, the resulting target DNA generally has a 5′-overhang or 3′-overhang, which may be converted to a blunt end in accordance with the methods as described in Step 2.

In Step 4 (Step 3 in case of the codon-based deletion mutagenesis of the target DNA), the blunt-ended both termini of the target DNA are subjected to self-ligation with a ligase to produce a library of randomly mutated DNA.

The library of mutant DNA thus obtained includes the mutant DNAs wherein a multiple of three consecutive nucleotides are inserted in the target DNA or deleted therefrom, at random sites of the target DNA. In such case, deletion of the same number of nucleotides as those inserted from the target DNA results in a substitution mutant library; deletion of nucleotides from the target DNA without insertion of nucleotides results in a deletion mutant library; and insertion of nucleotides without deletion of nucleotides of the target DNA results in an insertion mutant library. FIGS. 1 to 3 respectively illustrates examples of substitution, insertion and deletion mutagenesis of the present invention.

In Step 5 (Step 4 in case of the codon deletion mutagenesis of the target DNA), a directed evolution method is provided, said method comprising the steps of expressing the random mutant library of target DNA prepared in the above steps in an appropriate host cell; and selecting or screening the expressed polypeptides to obtain mutant polypeptides having a desired property and polynucleotides encoding same.

Specifically, Step 5 comprises inserting the double-stranded mutant DNA prepared in the previous step into an appropriate expression vector, introducing the resulting expression vector into a host cell to obtain a library consisting of a plurality of clones, expressing the polynucleotides contained in the clones to obtain a library of mutant proteins, and screening a protein having a desired property therefrom by a conventional method.

Suitable expression methods include methods of producing and accumulating a gene product in cells; secreting a gene product from a cell and accumulating them in a medium; secreting a gene product into the periplasm of the cells; and the like methods. In preparing the recombinant DNA library, any expression vector operable in a selected host cell may be employed. Exemplary vectors include conventional vectors such as phage, plasmid, phagemid, viral vector and artificial chromosomes known in the art. A method for constructing an expression vector is well known in the art, e.g., in Sambrook, J. et al., Molecular Cloning: A Laboratory Manual, 2^{nd} ed., (1989) Cold Spring Harbor Laboratory Press, N.Y. A suitable host cell may be transformed with the resulting expression vector. Suitable hosts for expressing the recombinant DNA include a bacterium such as E. coli, Bacillus subtilis and B. brevis, etc.; an Actinomyces such as Streptomyces lividans; a yeast such as Saccharomyces serevisiae; a fungus such as Aspergillus oryzae, A. nidulans and A. niger; an animal cell such as COS-7, CHO, Vero and mouse L cells; an insect cell; and a plant cell.

As the target DNA of the inventive mutagenesis method, there may be used any DNA encoding a protein and has been used for improving or evolving its properties by a conventional mutagenesis method. For instance, there have been reported mutants of various target proteins, e.g., enzymes, antibodies, antigens, binding proteins, hormones, cytokines and plasma proteins, developed by a conventional mutagenesis method (Lingen B. et al., Protein Eng. 15:585-593, 2002; Gaytan, P. et al., Nucleic Acids Res. 30:e84, 2002; Zhao, H. et al., Curr. Opin. Biotechnol. 13:104-110, 2002; Pikkemaat, M. G. and Janssen, D. B. Nucleic Acids Res. 30:E35-35, 2002; Santoro, S. W. and Schults, P. G. Proc. Natl. Acad. Sci. 99:4185-4190, 2002; Meyer, A. et al., J. Biol. Chem. 277:5575-5582, 2002; Wang, C. W. and Liao, J. C. J. Biol. Chem. 276:41161-41164, 2001; Suenaga, H. et al., J. Bacteriol. 183:5441-5444, 2001; Liebeton, K. et al., Chem. Biol. 7:709-718, 2000; Bettsworth, F. et al., J. Mol. Recognit. 14:99-109, 2001; Fares, F. A. et al., J. Biol. Chem. 276:4543-4548, 2001; Gulich, S. et al., J. Biotechnol. 76:233-244, 2000; Jung, S. et al., J. Mol. Biol. 19:163-180, 1999; Wu, H. et al., J. Mol. Biol. 294:151-162, 1999; Chen, G. et al., Protein Eng. 12:349-356, 1999; Wu, H. et al., Proc. Natl. Acad. Sci. 95:6037-6042, 1998; Ghetie, V. et al., Nat. Biotechnol. 15:637-640, 1997; Burks, E. A. et al., Proc. Natl. Acad. Sci. 94:412-417, 1997; Wong, Y. W. et al., J. Immunol. 154:3351-3358, 1995; Balint, R. F and Larrick, J. W., Gene 137:109-118, 1993; Rovinski, B. et al., Virology 257:438-448, 1999; Kurtzman, A. L. et al., Curr. Opin. Biotechnol. 12:361-370, 2001; Hu, R. et al., J. Immunol. 167:1482-1489, 2001; Plugariu, C. G., Biochemistry 39:14939-14949, 2000; Klein, B. K. et al., Exp. Hematol. 27:1746-1756, 1999; Gill, R. et al., Protein Eng. 9:1011-1019, 1996; Holler, P. D. Proc. Natl. Acad. Sci. 97:5387-5392, 2000; and Linskens, M. H. et al., FASEB J. 13:639-645, 1999) and, accordingly, the inventive mutagenesis methods can be applied to a DNA encoding such proteins for the directed evolution of such proteins. The DNA may encode an enzyme, said enzyme being selected from the group consisting of hydrolase, lyase, transferase, oxidoreductase, ligase and isomerase.

Once mutant proteins having desired properties are obtained by the screening of the mutant library, mutation sites therein can be confirmed by sequencing analysis of polynucleotides encoding them.

Further, it is possible to obtain a more improved mutant protein for the desired property by screening mutant polynucleotides having desired properties from the mutant library prepared by the inventive mutagenesis method; confirming the mutation sites therein by sequencing analysis; introducing two or more of the mutation sites existing in each mutant polynucleotide into one target DNA; and expressing and screening a mutant protein having more improved property. The process for introducing each mutation site existing in respective mutant polynucleotide into one target DNA can be conducted by conventional methods such as a method comprising digestion with a restriction enzyme and ligation of the resulting DNA; a sequential site-directed mutagenesis method; a polymerase chain reaction, and the like methods.

An improved mutant protein evolved for the desired purpose can be further obtained by repeatedly conducting the steps of constructing the mutant library by the inventive mutagenesis method using the mutant polynucleotide having two or more mutations therein as a target DNA and screening mutant polypeptides therefrom.

The random codon-based mutagenesis methods of the present invention have advantages over the conventional mutagenesis methods for improving the property of protein, as follows:

The inventive mutagenesis method may cause substitution by any possible amino acid at the respective amino acid residues of the polypeptide encoded by the target DNA.

Further, since the nucleotides introduced into a cassette DNA is inserted or substituted at only one site in the target DNA or consecutive nucleotides are randomly deleted at only one site of the target DNA by the inventive random mutagenesis method, the resulting target DNA will has a mutation at only one site. In this context, the inventive method is more convenient than the conventional random mutagenesis methods such as error-prone PCR and chemical mutagenesis, wherein beneficial mutations and deleterious mutations may occur simultaneously at plural places depending on the error-rate of the polymerase and, accordingly, the error-rate should be precisely controlled.

Furthermore, the inventive mutagenesis method is more economical than a site-specific mutagenesis method or a saturated mutagenesis method using oligonucleotides, because there is no need to synthesize every mutagenic oligonucleotides specific for each site of the target DNA. In addition, the inventive mutagenesis method is more convenient than the conventional methods since a mutant library can be prepared without a plurality of independent mutation reactions at respective sites of the target DNA. Further, since the inventive mutagenesis method does not require a mutagenic oligonucleotide to be bound to the target DNA, it can be easily used without the exact information on the nucleotide sequence of the target DNA.

Moreover, while the conventional methods for the directed evolution of a protein generally cause an amino acid substitution within the target polypeptide, the inventive mutagenesis method may cause not only substitution, but also insertion and deletion of amino acid at a random site and, therefore, it can be effectively used for a directed evolution of a target protein.

The present invention is further defined in the following Examples. It should be understood that these Examples, while indicating preferred embodiments of the invention by employing a specific enzyme, i.e., chitosanase, are given by way of illustration only. From the above discussion and these Examples, one skilled in the art can ascertain the essential characteristics of this invention, and without departing from the spirit and scope thereof, can make various changes and modifications of the invention to adapt it to various usage and conditions.

### EXAMPLE 1

### Preparation of a Substitution Mutant Library of Chitosanase Using the Inventive Random Codon-Based Mutagenesis Method

(Step 1) Insertion and Deletion of Transposon at a Random Site of Chitosanase Gene

Chitosanase gene (GenBank Accession No: AF334682) originating from Bacillus sp. KCTC 0377BP strain was amplified by PCR using a forward primer csn-n1 (5-AAAACTGCAGCATTTTATGTAGTAAGC-3; SEQ ID NO: 1) and a reverse primer csn-c1 (5-CCGGAATTCGTATGCTAATTCCC-3; SEQ ID NO: 2). The amplified DNA fragment was digested with PstI and EcoRI to obtain a DNA fragment of about 1.4-kb in size. The resulting DNA fragment was ligated to the PstI/EcoRI backbone of pUC19 (New England Biolabs) to give a recombinant plasmid designated pBC17.

To insert Tn7-based transposon into plasmid pBC17, GPS-LS linker-scanning system (New England Biolabs) was employed. Transprimer-5 (New England Biolabs) was inserted into plasmid pBC17 according to the manufacturer's instructions, and E. coli DH5α (Takara) was transformed with the resulting plasmid. Positive transformants were selected by culturing them on LB-agar plates supplemented with 20 μg/ml of kanamycin and 50 μg/ml of ampicillin at 37° C. for 18 hours. The colonies formed on the plates were collected and subjected to plasmid DNA extraction by Qiaprep Spin Miniprep method (Qiagen). The extracted plasmid DNA was treated with EcoRI and PstI and subjected to agarose gel electrophoresis. The DNA fragments of about 3.2-kb in size containing the transposon within the target DNA (chitosanase gene) were extracted from the gel and inserted into EcoRI/PstI backbone of pUC19 plasmid using T4 DNA ligase.

The resulting plasmids were treated first with PmeI (New England Biolabs) which recognizes both termini of the transposon to remove the transposon in the target DNA, and then with a calf intestinal phosphatase (Roche) to remove the phosphate residue from the PmeI treated 5′-end. Then, the DNA fragments of about 4.2-kb in size were extracted from 0.8% agarose gel using a Gel extraction kit (Qiagen). The resulting DNA was designated pBC17-ΔTn.

(Step 2) Removal of the Nucleotides Originating from the Transposon and Nucleotides of the Target DNA Duplicated During the Transposon Insertion at One of the Enzyme-Digested Ends of the Target DNA

In order to prepare a first cassette DNA to be inserted into the cut site of the target DNA, a forward primer bam-fp (5′-GGATCCTATGTATCCGCT CATGAGACAATAACC-3′; SEQ ID NO: 3) and a reverse primer del-rp (5′-GGCATTCTGCACTCTTCACCTAGATCCTTTTTGATCAG-3′; SEQ ID NO: 4) having phosphorylated 5′-ends were synthesized. PCR was conducted using pBC KS+ plasmid (Stratagene) as a DNA template and primers bam-fp and del-rp. The reaction mixture contained 15 ng of plasmid pBC KS+, 0.2 mM each dNTP, 2 mM MgSO₄, 10 mM KCl, 10 mM (NH₄)₂SO₄, 20 mM Tris-HCl (pH 8.8), 0.1% Triton X-100, 2 units of Vent DNA polymerase (New England BioLabs) and 0.5 μM each primer in a total volume of 100 μl. PCR was carried out on a PTC-101 thermal cycler (MJ Research) at 94° C. for 3 min; 94° C. for 30 sec, 58° C. for 30 sec, 72° C. for 1 min (30 cycles); and 72° C., 5 min. The PCR product was analyzed by 0.8% agarose gel electrophoresis, and DNA fragments of about 1.5-kb in size were extracted from the gel using a GENECLEAN kit (Bio 101).

The first cassette DNA thus purified was inserted into pBC17-ΔTn prepared in Step 2 using T4 DNA ligase. The ligation reaction mixture contained 1 μg of pBC17-Δ Tn DNA, 1 μg cassette DNA, 50 mM Tris-HCl (pH 7.6), 10 mM MgCl₂, 1 mM ATP, 1 mM DTT, 5% (w/v) polyethylene glycol-8000 and 3 units of ligase (Gibco BRL) in a total volume of 30 μl, and the ligation reaction was carried out at 25° C. for 12 hours. E. coli DH5α (Takara) was transformed with the reaction mixture, and positive transformants were selected by culturing them on LB-agar plates supplemented with 20 μg/ml of chloramphenicol at 37° C. for 18 hours. The colonies grown on the plates were collected and subjected to plasmid DNA extraction by Qiaprep Spin Miniprep method (Qiagen). The extracted plasmid DNAs were treated with BsgI, and 3′-overhang were removed by T4 DNA polymerase (New England Biolabs). The resulting plasmid DNAs were treated with BamHI and subjected to 1% agarose gel electrophoresis. The DNA fragments of about 4.2-kb in size, wherein the first cassette DNA was removed from the target DNA (chitosanase gene), were extracted from the gel and designated pBC17-Δ CAS1.

(Step 3) Insertion of Nucleotides and Removal of Transposon-Derived Nucleotides and Consecutive Three Nucleotides of the Target DNA from the Other Cut Terminus of the Target DNA

In order to prepare a second cassette DNA to be inserted into the cut site of the DNA fragment obtained in Step 2, a forward primer bsg-sf (5′-CGGGATCCTTCTGCACTATGTAT CCGCTCATGAGACAATAAAC-3′; SEQ ID NO: 5) and a reverse primer bsg-sr (5′-NNNACGTCAATTACGGATCCTGC ACTCTTCACCTAGATCCTTTTTGATC-3′; SEQ ID NO: 6) having phosphorylated 5′-ends were synthesized. PCR was conducted using plasmid pBC KS+ as a DNA template and primers bsg-sf and bsg-sr, under the same condition for the preparation of the first cassette DNA in Step 2. The PCR product was analyzed by 0.8% agarose gel electrophoresis, and DNA fragments of about 1.5-kb in size were extracted from the gel using a GENECLEAN kit (Bio 101).

The second cassette DNA thus purified was treated with BamHI and ligated with pBC17-ΔCAS1 prepared in Step 2 using T4 DNA ligase. The reaction mixture contained 1 μg of pBC17-ΔCAS1 DNA, 1 μg cassette DNA, 50 mM Tris-HCl (pH 7.6), 10 mM MgCl₂, 1 mM ATP, 1 mM DTT, 5% (w/v) polyethylene glycol-8000 and 3 units of ligase (Gibco BRL) in a total volume of 30 μl, and the ligation reaction was carried out at 25° C. for 12 hours. E. coli DH5α (Takara) was transformed with the reaction mixture, and positive transformants were selected by culturing them on LB-agar plates supplemented with 20 μg/ml of chloramphenicol at 37° C. for 18 hours. The colonies grown on the plates were collected and subjected to plasmid DNA extraction by Qiaprep Spin Miniprep method (Qiagen). The extracted plasmid DNA was cut with BsgI.

The resulting DNA fragment of about 4.1-kb in size, from which the second cassette DNA was removed, had 3′-overhangs. The DNA fragment was treated with T4 DNA polymerase (New England Biolabs) to convert its 3′-overhangs into blunt ends, thereby resulting in a mutant DNA wherein consecutive three nucleotides of the target DNA were substituted with random nucleotides at a random site of the target DNA. The reaction mixture contained 0.5 μg of BsgI treated cassette DNA, 50 Mm NaCl, 100 mM Tris-HCl (pH 7.9), 10 mM MgCl₂, 1 mM DTT, 100 mM each dNTP and 1 unit of T4 DNA polymerase in a total volume of 30 μl, the reaction was carried out at 12° C. for 20 min, and then, the reaction mixture was kept at 75° C. for 10 minutes to inactivate T4 DNA polymerase.

(Step 4) Preparation of a Mutant Library by Self-Ligation

The DNA fragment obtained in Step 3 was subjected to self-ligation using a DNA ligase. The reaction mixture contained 0.5 μg of the DNA fragment, 50 mM Tris-HCl (pH 7.6), 10 mM MgCl₂, 1 mM ATP, 1 mM DTT, 5% (w/v) polyethylene glycol-8000 and 1 unit of ligase (Gibco BRL) in a total volume of 30 μl, and the ligation reaction was carried out at 25° C. for 12 hours.

E. coli JM105 (Amersham Pharmacia Biotech) was transformed with the ligation mixture, and positive transformants were selected by culturing them on LB-agar plates supplemented with 100 μg/ml of ampicillin at 37° C. for 18 hours, which resulted in a random mutant library of chitosanase gene.

Five positive clones were randomly selected from the mutant library and plasmid DNAs were extracted therefrom by Qiaprep Spin Miniprep method (Qiagen). Nucleotide sequences thereof were analyzed in order to confirm the mutated sequence.

Table 1 shows the results of analyzing the nucleotide sequences of mutant genes randomly selected from the substitution mutant library of chitosanase gene. The underlined nucleotide represents a mutation site where nucleotide substitution occurred.

TABLE 1

| Mutant | Changes in amino acid and |
|---|---|
| chitosanase gene | codon nucleotide sequences |
| rcm-s1 | Val⁴²(GTT) → Glu⁴²(GAG) |
| rcm-s2 | Ala¹⁹(GCT) → Gly¹⁹(CCA) |
| rcm-s3 | Thr¹³¹(ACA), Val¹³²(GTA) → Asn¹³¹(AAT), |
| | Leu¹³²(CTA) |
| rcm-s4 | Trp³⁹⁷(TGG) → Gly³⁹⁷(GGG) |
| rcm-s5 | Ile³²⁷(ATT) → Asp³²⁷(GAC) |

### EXAMPLE 2

### Preparation of an Insertion Mutant Library of Chitosanase Gene Using the Inventive Random Codon-Based Mutagenesis Method

A mutant library having a random nucleotide sequences (NNN) inserted at a random site of a target DNA (chitosanase gene) was constructed according to the same method described in Example 1, except that a forward primer bsg-if (5′-CGGGATCCTTGCACTGCACTATGTATCCGCTCATGAGACAATAACC-3′; SEQ ID NO: 7) was employed for preparing a second cassette DNA in Step 3.

Four positive clones were randomly selected from the randomized insertion mutant library. Plasmid DNAs were extracted from the clones by Qiaprep Spin Miniprep method (Qiagen) and the nucleotide sequences thereof were analyzed.

Table 2 shows the results of analyzing the nucleotide sequences of said mutant genes. The ∇ mark represents a mutation site where randomized nucleotide insertion occurred, and the underlined nucleotide, inserted nucleotides.

TABLE 2

| Mutant | Changes in amino acid and |
|---|---|
| chitosanase gene | codon nucleotide sequences |
| rcm-i1 | Tyr³⁶⁵(TA^{∇}T) → Tyr³⁶⁵(TAT), His³⁶⁶(CAT) |
| rcm-i2 | Gln¹⁵⁹(CAA^{∇}) → Gln¹⁵⁹(CAA^{∇}), Ser¹⁶⁰(AGC) |
| rcm-i3 | Asn²³¹(AAT^{∇}) → Asn²³¹(AAT^{∇}), lLe²³²(ATA) |
| rcm-i4 | Tyr²⁷³(T^{∇}AC) → Ser²⁷³(TCC), Tyr²⁷⁴(TAC) |

### EXAMPLE 3

### Preparation of a Deletion Mutant Library of Chitosanase Gene Using a Random Codon-Based Mutagenesis Method

Tn7 transposon was inserted at a random site of chitosanase gene and removed according to the same method described in Step 1 of Example 1, to obtain chitosanase gene cut at a random site(pBC17-ΔTn).

To prepare a first cassette DNA to be inserted into the cut site of the DNA thus obtained, a forward primer bsg-df (5′-GCTACGCACTGCACTATGTATCC GCTCATGAGACAATAACC-3′; SEQ ID NO: 8) and a reverse primer bsg-dr (5′-GGCATTCTGCACTCTTCACCTAGATCCTTTTTGATCAG-3′; SEQ ID NO: 9) having a phosphorylated 5′-end were synthesized. PCR was conducted using plasmid pBC KS+ as a DNA template and primers bsg-df and bsg-dr according to the same method described in Step 3 of Example 1. The amplified cassette was analyzed by 0.8% agarose gel electrophoresis, and a DNA fragment of about 1.5-kb in size was extracted from the gel using a GENECLEAN kit (Bio 101).

The purified cassette DNA was inserted into plasmid pBC17-ΔTn using T4 DNA ligase. The reaction mixture contained 1 μg of pBC 17-ΔTn DNA, 1 μg cassette DNA, 50 mM Tris-HCl (pH 7.6), 10 mM MgCl₂, 1 mM ATP, 1 mM DTT, 5% (w/v) polyethylene glycol-8000 and 3 units of ligase (Gibco BRL) in a total volume of 30 μl, and a ligation reaction was carried out at 25° C. for 12 hours. E. coli DH5α (Takara) was transformed with the ligation mixture, and positive transformants were selected by culturing them on LB-agar plates supplemented with 20 μg/ml of chloramphenicol at 37° C. for 18 hours. The colonies formed on the plates were collected and subjected to plasmid DNA extraction by Qiaprep Spin Miniprep method (Qiagen). The extracted plasmid DNA was treated with BsgI to remove the cassette DNA.

The resulting DNA fragment of about 4.1-kb in size had 3′-overhangs, which was removed by treating with T4 DNA polymerase (New England Biolabs) to obtain blunt ends. The reaction mixture contained 0.5 μg of BsgI treated cassette DNA, 50 mM NaCl, 100 mM Tris-HCl (pH 7.9), 10 mM MgCl₂, 1 mM DTT, 100 mM each dNTP and 1 unit of T4 DNA polymerase in a total volume of 30 μl, and the conversion reaction was carried out at 12° C. for 20 min, and then, the reaction mixture was kept at 75° C. for 10 min to inactivate T4 DNA polymerase.

The resulting DNA was subjected to self-ligation using DNA ligase according to the method described in Step 4 of Example 1, which resulted in a random-deletion mutant library of chitosanase gene.

Five positive clones were randomly selected from the mutant library. Plasmid DNAs were extracted from the clones by Qiaprep Spin Miniprep method (Qiagen) and the nucleotide sequences thereof were analyzed.

Table 3 shows the results of analyzing the nucleotide sequences of mutant genes. The underlined nucleotide represents a site where nucleotide deletion occurred, and the Δ mark, site where the deletion of corresponding amino acid occurred.

TABLE 3

| Mutant | Changes in amino acid and |
|---|---|
| chitosanase gene | codon nucleotide sequences |
| rcm-d1 | Trp³⁹⁷(TGG) → ΔTrp³⁹⁷ |
| rcm-d2 | Arg³³⁸(AGA) → ΔArg³³⁸ |
| rcm-d3 | Gln⁷²(CAG), Glu⁷³(GAA) → Gln⁷²(CAA), ΔGlu⁷³ |
| rcm-d4 | Gly¹²⁵(GGG), Tyr¹²⁶(TAT) → Gly¹²⁵(GGT), ΔTyr¹²⁶ |

### EXAMPLE 4

### Screening of Mutant Chitosanase Having an Enhanced Thermostability

Chitosanase having an enhanced thermostability was selected from the substitution mutant library prepared in Example 1, as follows.

E. coli DH5α (Takara) was transformed with the mutant library, and positive transformants obtained from the library were replica-plated onto LB-agar plates supplemented with 100 μg/ml of ampicillin at 37° C. for 20 hours. The petri dish containing the colonies was heated on a water bath at 70° C. for 15 min, and then 50 mM Na-acetate buffer solution containing 0.1% chitosan and 1% agarose was poured onto the LB-agar plates. After the plates were kept at 37° C. for 24 hours, colonies still having the activity to produce clear plaques were selected using 0.2% Congo Red. As a result, three positive clones having improved thermostability were isolated. Plasmid DNAs were extracted from the clones by Qiaprep Spin Miniprep method (Qiagen), and the nucleotide sequences of the thermally stable chitosanase genes were analyzed.

Table 4 shows the amino acid substitution sites of the thermostable chitosanase mutants. The underlined nucleotide represents a mutation site where an amino acid substitution occurred.

TABLE 4

| Mutant | Changes in amino acid and |
|---|---|
| chitosanase gene | codon nucleotide sequences |
| rcm-t1(N368E) | Asn³⁶⁸(AAT) → Glu³⁶⁸(GAG) |
| rcm-t2(N297S) | Asn²⁹⁷(AAC) → Ser²⁹⁷(AGT) |
| rcm-t3(Q159R) | Gln¹⁵⁹(CAA) → Arg¹⁵⁹(CGT) |

The above identified mutation sites confirmed by sequencing analysis of the mutant chitosanase genes were introduced into a single chitosanase gene by successive site-specific mutagenesis method using QuickChange site-directed mutagenesis kit (Stratagene). E. coli DH5α (Takara) was transformed with the triple mutant thus obtained or the respective single mutants and the positive transformants were cultured in a LB broth at 37° C. for 24 hours. The resulting culture solution was centrifuged to obtain a supernatant and the thermostability of each mutant chitosanase contained therein was measured.

Table 5 shows the activity of each mutant chitosanase remained after treatment at 55° C. for 30 minutes in comparison with that of a wild-type chitosanase.

TABLE 5

| | Remaining |
|---|---|
| Chitosanase | activity (%) |
| Wild-type | 43.2 |
| rcm-t1(N368E) | 78.2 |
| rcm-t2(N297S) | 54.3 |
| rcm-t3(Q159R) | 74.6 |
| rcm-t4(N378E + N297S + Q159R) | 94.2 |

This result shows that the thermal stability of the inventive mutant, the triple mutant in particular, is markedly higher than that of the wild-type chitosanase.

As can be appreciated from the disclosure and the examples above, the method of the present invention can be used for directed molecular evolution to obtain proteins having desired properties and polypeptides encoding same.

While the invention has been described with respect to the above specific embodiments, it should be recognized that various modifications and changes may be made to the invention by those skilled in the art which also fall within the scope of the invention as defined by the appended claims.

1. A method for evolving a polypeptide and a polynucleotide encoding same by random substitution of nucleotides, comprising the steps of:
1) inserting a transposon having restriction enzyme sites on both ends thereof into a random position of a double-stranded target DNA, introducing the resulting DNA into a circular DNA construct and cutting the transposon at the restriction enzyme sites to remove the transposon and obtain a linearized DNA construct containing two cut termini of the target DNA cut in one position;
2) deleting the nucleotides originating from the transposon and the nucleotides of the target DNA duplicated during the insertion of the transposon, at one cut terminus of the target DNA;
3) inserting a multiple of three substitutive nucleotides into one cut terminus of the target DNA subjected to deletion in Step 2, and deleting the nucleotides originating from the transposon and a multiple of three consecutive nucleotides of the target DNA at the other cut terminus of the target DNA obtained in Step 1;
4) subjecting both cut termini of the target DNA obtained in Step 3 to self-ligation to obtain a library of mutant DNA having substitutive nucleotides at a random position; and
5) expressing the resulting library in an appropriate host cell and selecting or screening the expressed-polypeptides to obtain a mutant polypeptide having a desired property and a polynucleotide encoding same.

2. The method of claim 1, wherein Step 2 comprises the steps of introducing a first cassette DNA into the cut position of the target DNA, digesting the cassette DNA with a restriction enzyme, and converting the cut terminus having nucleotides duplicated during the insertion of the transposon to a blunt end, thereby resulting in the deletion of the nucleotides originating from the transposon and the nucleotides of the target DNA duplicated during the insertion of the transposon.

3. The method of claim 1, wherein Step 3 comprises the steps of introducing a second cassette DNA containing a multiple of three consecutive substitutive nucleotides into the cut position of the DNA obtained in Step 2, digesting the second cassette DNA with a restriction enzyme, and converting both cut termini of the resulting DNA fragment to blunt ends, thereby resulting in the addition of the substitutive nucleotides into one cut terminus of the target DNA subjected to deletion in Step 2 and the deletion of the nucleotides originating from the transposon and a multiple of three consecutive nucleotides of the target DNA at the other cut terminus of the target DNA obtained in Step 1.

4. The method of claim 1, wherein the transposon is selected from the group consisting of Tn4430, Tn7, mini-Mu and derivatives thereof.

5. The method of claim 1, wherein the substitutive nucleotides introduced in Step 3 have a specific nucleotide sequence.

6. The method of claim 1, wherein the substitutive nucleotides introduced in Step 3 have a random nucleotide sequence.

7. The method of any one of claims 1 to 6, wherein the target DNA encodes a protein selected from the group consisting of enzymes, antibodies, antigens, binding proteins, hormones, cytokines and plasma proteins

8. The method of claim 7, wherein the enzyme is selected from the group consisting of hydrolase, lyase, transferase, oxidoreductase, ligase and isomerase.

9. The method of claim 2 or 3, wherein the restriction enzyme is a class IIS restriction enzyme.

10. A method for evolving a polypeptide and a polynucleotide encoding same by random insertion of nucleotides, comprising the steps of:
1) inserting a transposon having restriction enzyme sites on both ends thereof into a random position of a double-stranded target DNA, introducing the resulting DNA into a circular DNA construct and cutting the transposon at the restriction enzyme sites to remove the transposon and obtain a linearized DNA construct containing two cut termini of the target DNA cut in one position;
2) deleting the nucleotides originating from the transposon and the nucleotides of the target DNA duplicated during the insertion of the transposon, at one cut terminus of the target DNA;
3) inserting a multiple of three additional nucleotides into one cut terminus of the target DNA subjected to deletion in Step 2, and deleting the nucleotides originating from the transposon at the other cut terminus of the target DNA obtained in Step 1;
4) subjecting both cut termini of the target DNA obtained in Step 3 to self-ligation to obtain a library of mutant DNA having additional nucleotides at a random position; and
5) expressing the resulting library in an appropriate host cell and selecting or screening the expressed polypeptides to obtain a mutant polypeptide having a desired property and a polynucleotide encoding same.

11. The method of claim 10, wherein Step 2 comprises the steps of introducing a first cassette DNA into the cut position of the target DNA, digesting the cassette DNA with a restriction enzyme, and converting the cut terminus having nucleotides duplicated during the insertion of the transposon to a blunt end, thereby resulting in the deletion of the nucleotides originating from the transposon and the nucleotides of the target DNA duplicated during the insertion of the transposon.

12. The method of claim 10, wherein Step 3 comprises the steps of introducing a second cassette DNA containing a multiple of three consecutive additional nucleotides into the cut position of the DNA obtained in Step 2, digesting the second cassette DNA with a restriction enzyme, and converting both cut termini of the resulting DNA fragment to blunt ends, thereby resulting in the insertion of the additional nucleotides into one cut terminus of the target DNA subjected to deletion in Step 2, and the deletion of the nucleotides originating from the transposon at the other cut terminus of the target DNA obtained in Step 1.

13. The method of claim 10, wherein the transposon is selected from the group consisting of Tn4430, Tn7, mini-Mu and derivatives thereof.

14. The method of claim 10, wherein the additional nucleotides introduced in Step 3 have a specific nucleotide sequence.

15. The method of claim 10, wherein the additional nucleotides introduced in Step 3 have a random nucleotide sequence.

16. The method of any one of claims 10 to 15, wherein the target DNA encodes a protein selected from the group consisting of enzymes, antibodies, antigens, binding proteins, hormones, cytokines and plasma proteins

17. The method of claim 16, wherein the enzyme is selected from the group consisting of hydrolase, lyase, transferase, oxidoreductase, ligase and isomerase.

18. The method of claim 11 or 12, wherein the restriction enzyme is a class IIS restriction enzyme.

19. A method for evolving a polypeptide and a polynucleotide encoding same by random deletion of nucleotides, comprising the steps of:
1) inserting a transposon having restriction enzyme sites on both ends thereof into a random position of a double-stranded target DNA, introducing the resulting DNA into a circular DNA construct and cutting the transposon at the restriction enzyme sites to remove the transposon and obtain a linearized DNA construct containing two cut termini of the target DNA cut in one position;
2) deleting the nucleotides originating from the transposon and the nucleotides of the target DNA duplicated during the insertion of the transposon, at one cut terminus of the target DNA, and the nucleotides originating from the transposon and a multiple of three consecutive nucleotides of the target DNA at the other cut terminus of the target DNA obtained in Step 1;
3) subjecting both cut termini of the target DNA obtained in Step 2 to self-ligation to obtain a library of mutant DNA having a deletion of nucleotides at a random position; and
4) expressing the resulting library in an appropriate host cell and selecting or screening the expressed polypeptides to obtain a mutant polypeptide having a desired property and a polynucleotide encoding same.

20. The method of claim 19, wherein Step 2 comprises the steps of introducing a cassette DNA into the cut position of the target DNA, digesting the cassette DNA with a restriction enzyme, and converting both cut termini of the resulting DNA fragment to blunt ends, thereby resulting in the deletion of the nucleotides originating from the transposon and the nucleotides of the target DNA duplicated during the insertion of the transposon, at one cut terminus of the target DNA, and the deletion of the nucleotides originating from the transposon and a multiple of three consecutive nucleotides of the target DNA at the other cut terminus of the target DNA obtained in Step 1.

21. The method of claim 19, wherein the transposon is selected from the group consisting of Tn4430, Tn7, mini-Mu and derivatives thereof.

22. The method of any one of claims 19 to 21, wherein the target DNA encodes a protein selected from the group consisting of enzymes, antibodies, antigens, binding proteins, hormones, cytokines and plasma proteins

23. The method of claim 22, wherein the enzyme is selected from the group consisting of hydrolase, lyase, transferase, oxidoreductase, ligase and isomerase.

24. The method of claim 20, wherein the restriction enzyme is a class IIS restriction enzyme.

25. A method for evolving a polypeptide and a polynucleotide encoding same, comprising the steps of:
1) preparing a library of mutant polynucleotides having a plurality of mutations by introducing two or more mutated sequences identified in two or more mutant polynucleotides selected by at least one of the methods of claims 1, 10 and 19, into a target polynucleotide; and
2) expressing the library obtained in Step 1 in an appropriate host cell and selecting or screening the expressed polypeptides to obtain a mutant polypeptide having a desired property and a polynucleotide encoding same.

26. A method for evolving a polypeptide and a polynucleotide encoding same, comprising repeating the method of any one of claims 1, 10 and 19 with the mutant polynucleotide prepared by the method of claim 25 as a target polynucleotide.
